(19) United States
(12) Patent Application Publication
(10) Pub. No.: US 2017/0224538 A1
(43) Pub. Date: Aug. 10, 2017

(54) Bandage For Applying Arterial Pressure

(71) Applicants: Barakat Alhammadin, Brea, CA (US); Christopher Abboud, San Diego, CA (US)

(72) Inventors: Barakat Alhammadin, Brea, CA (US); Christopher Abboud, San Diego, CA (US)

(21) Appl. No.: 15/494,794

(22) Filed: Apr. 24, 2017

Related U.S. Application Data
(63) Continuation-in-part of application No. 14/287,367, filed on May 27, 2014.

(51) Int. Cl. A61F 13/02; A61F 13/00

(52) U.S. Cl. CPC A61F 13/0233; A61F 13/00068; A61F 13/0243; A61F 2013/0028

(57) ABSTRACT

A hemostasis device comprises a panel having a top side and a bottom side having an adhesive layer coupled to the panel. A button, having a 3-dimensional shape, is integrally molded to the top side of the panel. One or more apertures extend through the button and the panel. A pad joined to the panel by the adhesive layer. The button is engaged to a wound by a user and adapted to provide a force vector in a substantially axial direction to the wound. A method for imparting hemostasis comprises the steps of selecting an arterial pressure device then positioning the button with the one or more apertures over a wound. Retaining the device over the wound imparts a force vector in a substantially axial direction to the wound and results in more rapid blood coagulation.

### CROSS-REFERENCE TO RELATED APPLICATION(S)

The present application is a continuation-in-part of, and claims priority to U.S. Utility patent application Ser. No. 14/287,367 filed on May 27, 2014, entitled “Bandage For Applying Arterial Pressure” the entire disclosure of which is incorporated by reference herein.

### BACKGROUND OF THE INVENTION

1. Field of Invention

The present invention relates to the field of bandages, more specifically, bandages for applying arterial pressure.

2. Description of Related Art

Generally, bandages have been used to protect open wounds from infection and further damage post-trauma. Bandages also help collect blood it has time to coagulate. To promote coagulation and prevent the excess loss of blood, the victim or medical aid will apply pressure to the wound, typically using their hands or a tourniquet. Each of these require the use of the victims hands, or a medical aid which may not be available at the time of trauma.

Hemostasis is accomplished via three mechanisms. First, vasoconstriction is the blood vessels first response to injury. Vasoconstriction is promoted by the smooth muscle cells, which are in turn controlled by the vascular endothelium. Collagen is exposed at the site of injury promoting platelets to adhere to the injury site. Second, platelets adhere to the damaged endothelium to form a platelet plug. The platelets then adhere to collagen and further produce glycoprotein receptors that interact with other platelets, promoting aggregation and adhesion. Third, clots form upon the conversion of fibrinogen to fibrin and its addition to the platelet plug. As the fibrin mesh forms, blood is transformed from a liquid to a gel.

To aid in the clotting process, pressure should be added to the wound post-trauma. Applying pressure to the wounded area causes the walls of the blood vessels to cave inward toward one another. This slows the turbulent flow of blood and increases the rate of coagulation at the wounded area. In the event of a gunshot wound, pressure must be applied to the wound until care at a hospital is received, or else the victim may die from exsanguination. The invention described herein elegantly addresses the need for constant pressure during trauma while reducing the need for a victim or caregiver to actively apply pressure to the wound.

The above process takes time while occupying the hands of one or more victims and medical aids. A suitable solution is desired.

Based on the foregoing, there is a need in the art for a bandage that applies arterial pressure to a wound, thus promoting hemostasis.

### SUMMARY OF THE INVENTION

An embodiment of the disclosure meets the needs presented above by generally comprising a panel that may be positioned over a wound. An adhesive layer is coupled to the panel. The adhesive layer engages a users skin. The panel is retained on the wound. A button is couple to the panel. The button is engaged by the user. The button transfers direct pressure to the wound. The button stops bleeding of the wound.

An object of the invention is to provide a device that is a bandage for applying arterial pressure.

These together with additional objects, features, and advantages of the bandage for applying arterial pressure will be readily apparent to those of ordinary skill in the art upon reading the following detailed description of presently preferred, but nonetheless illustrative, embodiments of the bandage for applying arterial pressure when taken in conjunction with the accompanying drawings.

In this respect, before explaining the current embodiments of the bandage for applying arterial pressure in detail, it is to be understood that the bandage for applying arterial pressure is not limited in its application to the details of construction and arrangements of the components set forth in the following description or illustration. Those skilled in the art will appreciate that the concept of this disclosure may be readily utilized as a basis for the design of other structures, method, and systems for carrying out the several purposes of the bandage for applying arterial pressure.

It is therefore important that the claims be regarded as including such equivalent construction insofar as they do not depart from the spirit and scope of the bandage for applying arterial pressure. It is also to be understood the phraseology and terminology employed herein are for purposes of the description and should not be regarded as limiting.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a perspective view of the bandage assembly, according to an embodiment of the present invention;

FIG. 2 is an exploded perspective view of the device, according to an embodiment of the present invention;

FIG. 3 is a bottom view of the device, according to an embodiment of the present invention;

FIG. 4 is a cross-sectional view of the device, according to an embodiment of the present invention;

FIG. 5 is an in-use view of the device, according to an embodiment of the present invention;

FIG. 6 is an top plan view of the bandage assembly, according to an embodiment of the present invention;

FIG. 7 is an top view of the device, according to an embodiment of the present invention;

FIG. 8 is an side elevation view of the device, according to an embodiment of the present invention;

FIG. 9 is an exploded perspective view of the device, according to an embodiment of the present invention;

FIG. 10 is a side elevation view of the device, according to an embodiment of the present invention;

FIG. 11 is a perspective view of the device, according to an embodiment of the present invention;

FIG. 12 is a top plan view of the device having a hexagonal button, according to an embodiment of the present invention;

FIG. 13 is a perspective view illustrating the flexible qualities of the device, according to an embodiment of the present invention;

FIG. 14 is a perspective view of the device, according to an embodiment of the present invention; and

FIG. 15A is a perspective view of the device to be used on a small appendage, according to an embodiment of the present invention.

FIG. 15B is a front elevation view of the device to be used on a small appendage, according to an embodiment of the present invention.

FIG. 16A is a front elevation view of the to be used in the event of mass-trauma such as a gunshot wound, according to an embodiment of the present invention.

FIG. 16B is a perspective view of the device to be used in the event of mass-trauma such as a gun shot wound, according to an embodiment of the present invention;

FIG. 17 is a flowchart of a method of use, according to an embodiment of the present invention; and

FIG. 18 is a flowchart of a method of use in a mass-trauma scenario such as a gunshot wound, according to an embodiment of the present invention.

### DETAILED DESCRIPTION OF PREFERRED EMBODIMENTS

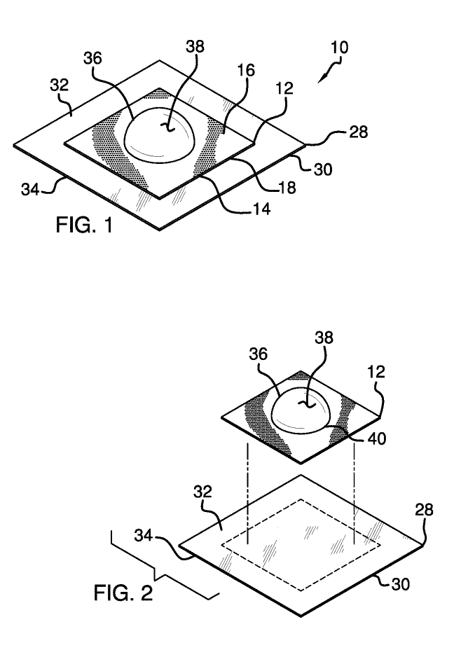

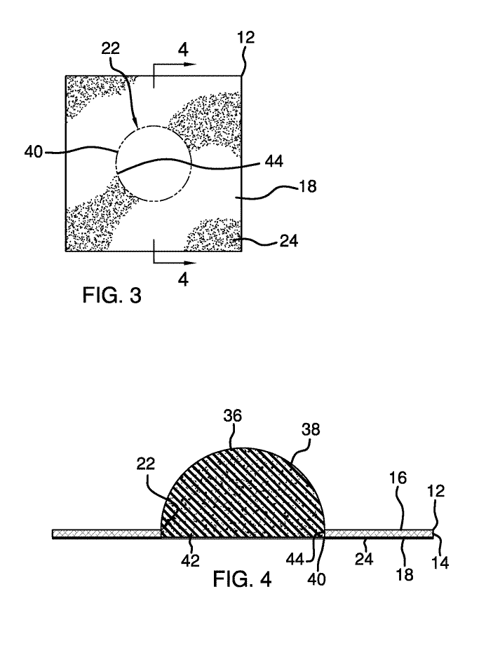

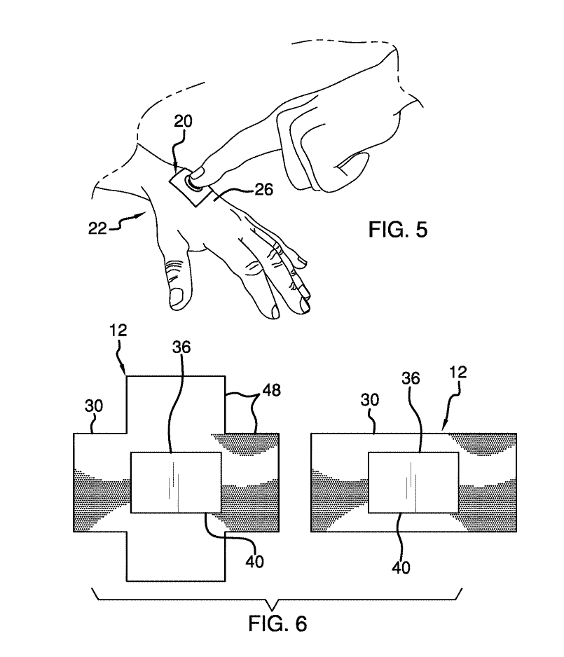

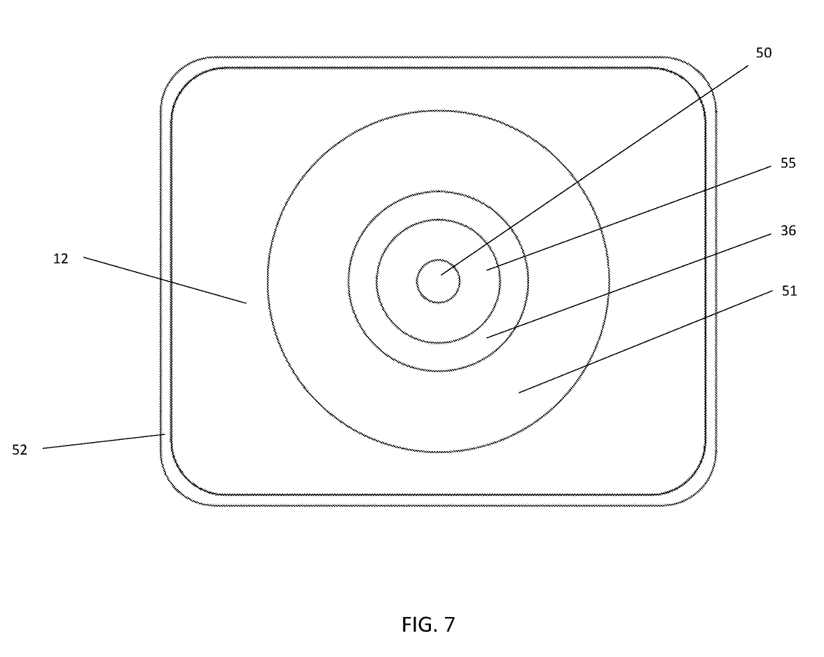

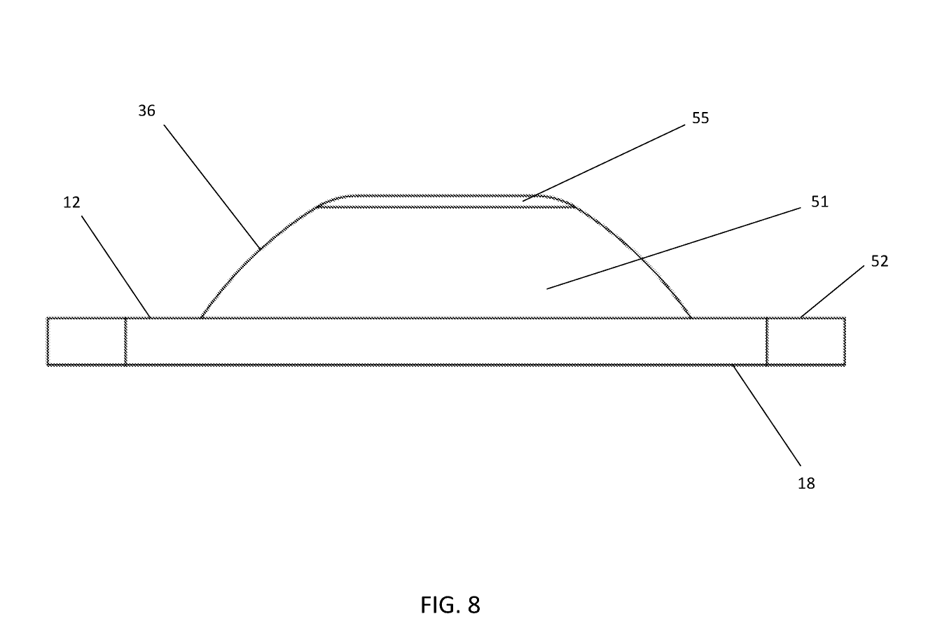

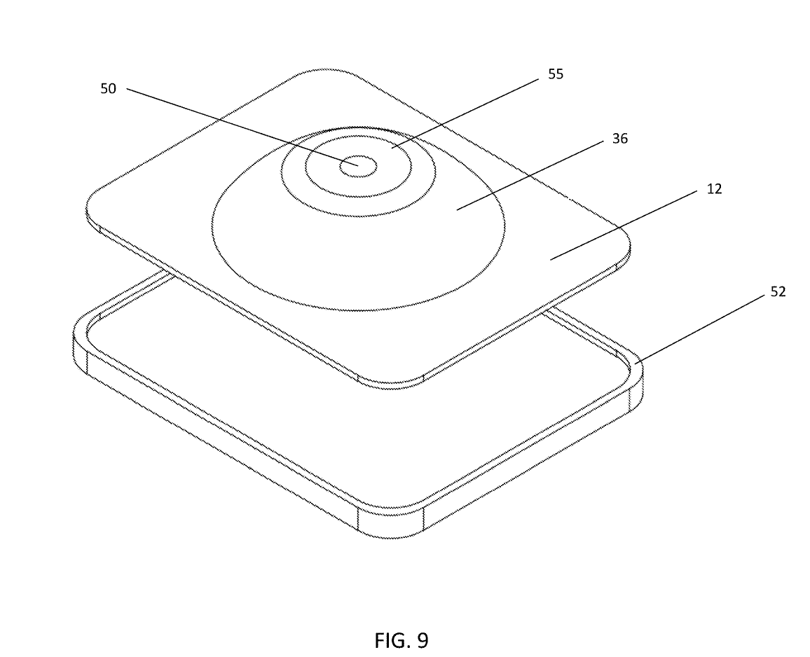

Preferred embodiments of the present invention and their advantages may be understood by referring to FIGS. 1-18, wherein like reference numerals refer to like elements.

As best illustrated in FIGS. 1 through 6, the bandage assembly 10 generally comprises a panel 12 that has an exterior edge 14 extending between a top side 16 and a bottom side 18 of the panel 12. The panel 12 may be positioned over a wound 20 on a user 21. The wound 20 may be an arterial wound or a venous wound. Moreover the exterior edge 14 of the panel 12 has a width that is similar to a length of the exterior edge 14 of the panel 12. The panel 12 may have a square shape. The panel 12 has a button opening 22 extending through the top 16 and bottom 18 sides of the panel 12.

An adhesive layer 24 is coupled to the bottom side 18 of the panel 12. The adhesive layer 24 completely covers the bottom side 18 of the panel 12. Additionally, the adhesive layer 24 engages the users skin 26 so the panel 12 is retained on the wound 20. The adhesive layer 24 may be comprised of a non-residual medical grade adhesive of any conventional design.

A pad 28 has an outer edge 30 extending between an upper side 32 and a lower side 34 of the pad 28. The pad 28 is removably coupled to the adhesive layer 24. Moreover, the pad 28 is peeled away from the adhesive layer 24 when the panel 12 is to be applied to the wound 20. The pad 28 protects the adhesive layer 24 until the panel 12 is utilized.

A button 36 is provided. The button 36 has a top surface 38 extending upwardly from an extraneous edge 40 of a lowermost side 42 of the side button 36. The top surface 38 of the button 36 is curvilinear. Moreover, the button 36 has a hemispherical shape.

The button 36 is positioned within the button opening 22. The lowermost side 42 of the button 36 lies on a plane that is planar with the bottom side 18 of the panel 12. Additionally, the extraneous edge 40 of the lowermost side 42 of the button 36 is coupled to a bounding edge 44 of the button opening 22. The button 36 is retained on the panel 12.

The top surface 38 of the button 36 is positioned over the wound and is depressed by the user 21 after applying force to the panel 12. The button 36 transfers direct pressure to the wound 20. Additionally, the button 36 stops the wound 20 from bleeding.

The button 36 may be comprised of a resiliently compressible material.

In an alternative embodiment, the outer edge 30 of the panel 12 may define a pair of intersecting arms 48 of the panel 12. The panel 12 may have a cross shape. Additionally, the outer edge 30 of the panel may have a length that is greater than a width of the outer edge 30 of the panel 12. The panel 12 may have a rectangular shape. The extraneous edge 40 of the lowermost side 42 of the side button 36 may have a width that is greater than a length of the extraneous edge 40 of lowermost side 42 the button 36. The button 36 may have a rectangular shape.

In use, the pad 28 is removed from the adhesive layer 24. The panel 12 is positioned over the wound 20 so the top surface 38 of the button 36 engages the wound 20. The user 21 depresses the button 36 so the top surface 38 of the button 36 transfers the direct pressure to the wound 20. The user 21 continues to depress the button 36 until the bleeding is controlled or until the user 21 is able to seek medical attention.

With respect to the above description, it is to be realized that the optimum dimensional relationship for the various components of the bandage assembly 10, to include variations in size, materials, shape, form, function, and the manner of operation, assembly and use, are deemed readily apparent and obvious to one skilled in the art, and all equivalent relationships to those illustrated in the drawings and described in the specification are intended to be encompassed by the bandage assembly 10.

In reference to FIGS. 6 through 9, the button 36 further comprises one or more apertures 50 positioned through the central axis of the button 36. The through-hole is configured to receive medication, a needle, or other means of medical intervention without the user removing the bandage assembly. In a preferred embodiment, the aperture 50 receives an absorbent material such as gauze. The plurality of apertures 50 allow for multiple uses such as one or more being used for absorbent material, and another for a needle or insertion of medication among other medical interventions known in the art. In a preferred embodiment and in use, the aperture 50 is positioned directly over the wound such that discharged fluids are collected by absorbent material within the aperture 50.

In an embodiment, the pad 28 comprises an elevated perimeter 52, which is dimensioned to retain the bottom side 18 of the panel 12.

In an embodiment, the button 36 is releasably engaged with the pad 28, allowing for the user to substitute alternate embodiments of the button 36 design, shape, hardness, size and likewise characteristics. The button 36 may be retained within the elevated perimeter of the pad 28 by a friction fit or adhesive.

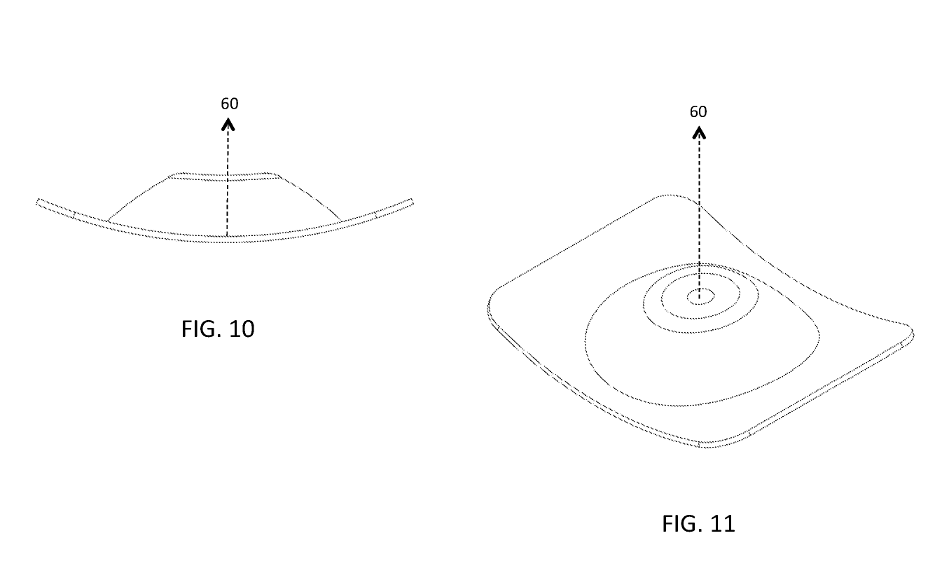

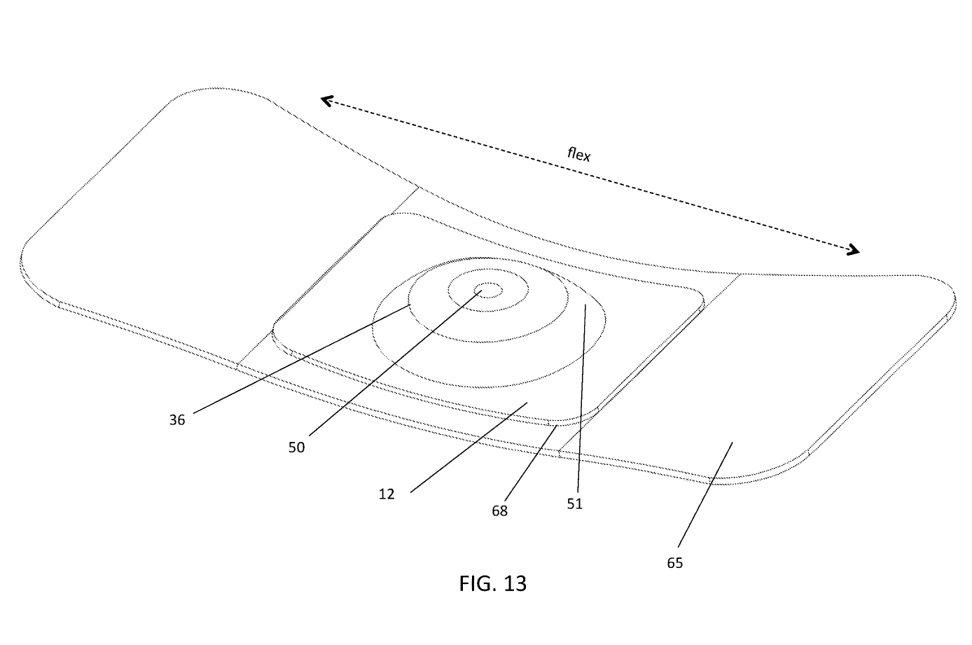

In an embodiment, the button 36 is a semi-dome having a flat top 55. The flat top 55 functions to increase the surface area, wherein the wound is under direct and even pressure when applied. In an embodiment, a beveled portion 51 separates the flat top 55 and the panel 12. In alternate embodiments, the button 36 may be any 3-dimensional shape as seen in FIG. 13. Substituting shapes of button 36 modifies the vector force 60 (shown in FIG. 10) and area over which the force is applied.

In use, as the user positions the button 36 over the wound, the pressure will compress the air out of the aperture due to the semi-resilient material of the dome. The evacuation of air from the button 36 creates a vacuum, drawing fluid from the wound into the aperture wherein lies absorbent material. Drawing fluid from the wound aids in the clotting process without the use of any chemicals.

In an embodiment, and in reference to FIGS. 10 and 11, the user may apply the device onto a wound, imparting a force vector 60 in a substantially axial direction in reference to the wound. The user may then position absorbent material such that the device is between the absorbent material and the wound. Pressure imparted by the button 36 creates a vacuum and draws fluid through the aperture 50. The fluid is then absorbed by the absorbent material.

In an embodiment, the pad 28 is a resilient acrylonitrile butadiene styrene (ABS) plastic, however other polymers known in the art may be used. In an embodiment, the durometer, as measured by Shore hardness, of the ABS plastic can be adjusted to yield a more or less flexible final product. The flexibility of the pad permits the device to be positioned over a wound and conform to the body's natural curvature. The flexibility of the pad directs force in a substantially axial direction to the wound, maintaining adequate arterial pressure. Varying the dimensions of the pad 28 increases or decreases the ability of the pad 28 to bend about the perimeter of the button. In reference to FIG. 10, the flexibility of the device imparts a greater force vector 60 onto the user.

In an embodiment, the structural rigidity of the device is defined by the infill density percentage of a 3D printer. The infill density ranges from 10% to 90% with 10% yielding a soft and malleably material and 90% yielding a hard and highly resilient material. In an embodiment wherein 3D printing is not used, a Shore hardness test may be used to determine the hardness and resilience of the pad, button, and other polymer materials.

In an embodiment, Shore Hardness defines the hardness of the material used for device. A range of 10 A to 95 A Shore Hardness may be used. In a preferred embodiment, a shore hardness of 85 A is used to apply the appropriate pressure onto the wound and allow for adequate flexibility of the material and the device in its entirety.

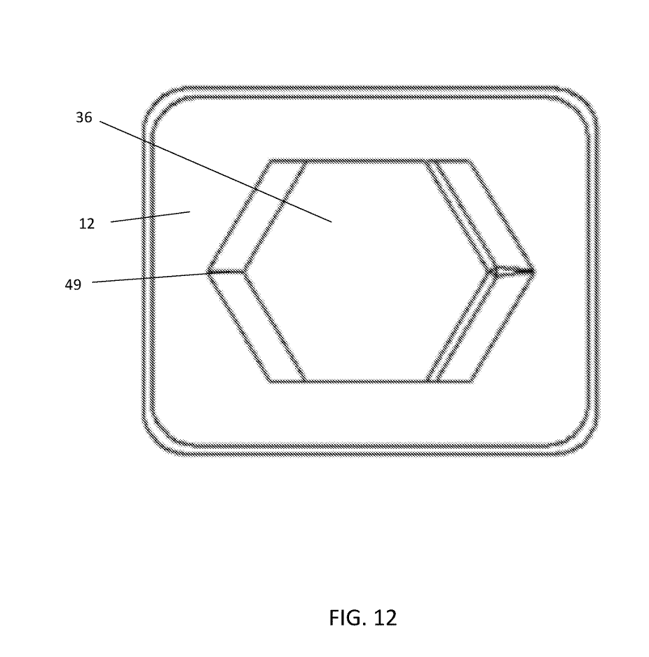

FIG. 12 illustrates an embodiment wherein the button is modified to a hexagonal 3-dimensional shape. During production, any 3-dimensional shape may be used such that the spirit of the invention remains. Altered 3-dimensional shapes can be used to better impress upon specific wounds. Rigid members 49 may be disposed within the 3-dimensional shape to promote directional flexibility of the panel 12 when applied to the wound. Alternate shapes serve to modify and focus the directional pressure of the button onto the wound.

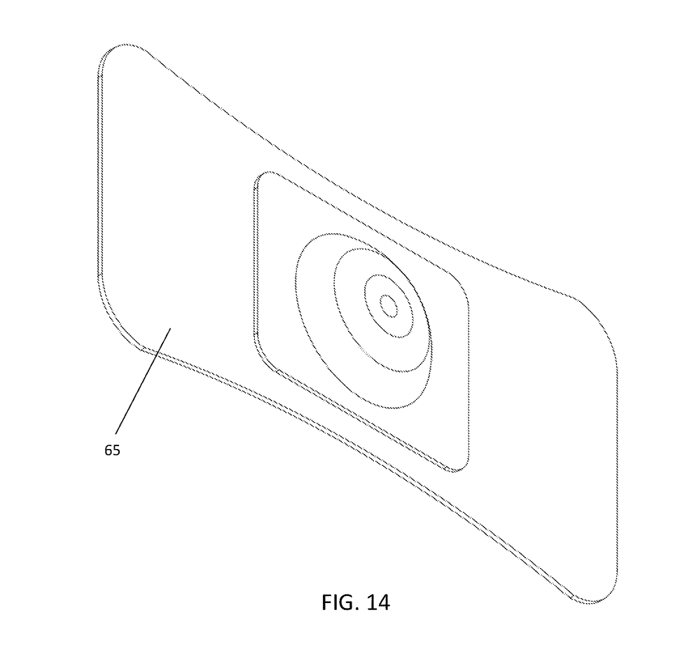

In reference to FIGS. 13 and 14, the arterial pressure device is positioned within a bandage 65 to aid in emitting additional pressure to a wound. The arterial pressure device may be positioned within two or more layers of the bandage 65 or coupled at an end of the bandage 65. Additionally, the arterial pressure device may be adhered to the interior side of the bandage. One or more flexible members 68 may be positioned between the top side 16 and bottom side 18. The flexible members 68 may allow the device to gain more desirable flexible characteristics. For example, the flexible members 68 may place directional focus pressure onto a wound. Flexible members may have a different durometer than the panel 12.

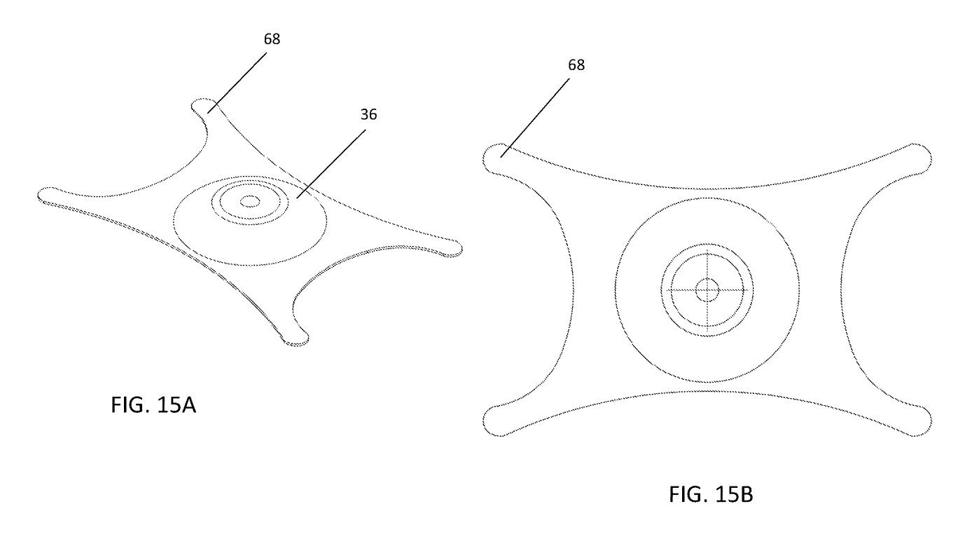

Referring now to FIGS. 15A and 15B, the device is shown having a panel 12 with protrusions 70 at each corner. This embodiment may be applied to a finger or other area of the body where increased flexibility of the device is desired. The protrusions are inherently flexible allowing for the panel 12 to bend around the appendage and ensure it remains in place while maintaining pressure on the wound. The button 36 may be positioned as illustrated, or may conform to the profile of the protrusions 70 providing an increase in pressure over the entire surface area of the panel 12.

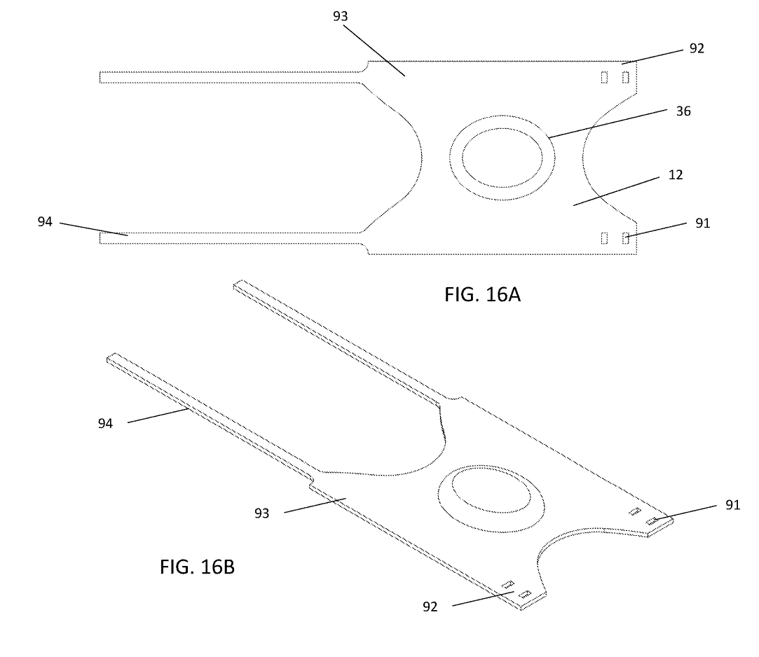

In reference to FIGS. 16A and 16B, the device is show in an alternate embodiment to preferentially be used for application with gunshot wounds or similar mass-trauma event. One or more arms 94 extend from a first side 93 of the panel 12. The arms 94 are comprised of a flexible material such as silicone, textile fabric, leather, or a material with similar malleable characteristic. One or more corresponding receivers 91 are disposed on the receiving side 92 of the panel 12. The panel 12 may be comprised of a malleable material such as silicone, textile fabric, leather, or similar material to permit increased range of motion of the material. Each receiver 91 retains one of the one or more arms 94 around the body of the user such that adequate pressure is maintained in a substantially axial direction to the wound.

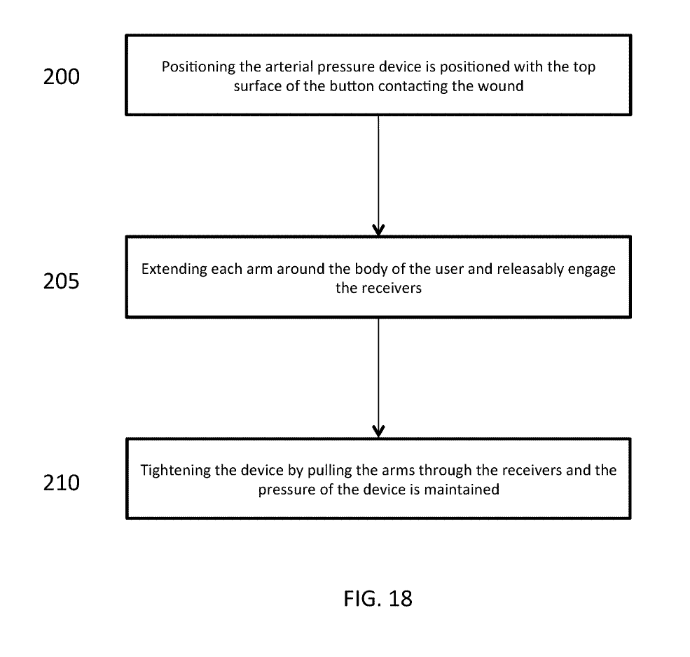

In reference to FIG. 18, a method of hemostasis following a mass-trauma event is illustrated, wherein the embodiment illustrated in FIGS. 16A and 16B is used. In step 200, a user positions the button of a device over the wounded area. In step 205, the arms encircle the user's body and releasably engage the receivers. In step 210, the user tightens the device to instill direct pressure in a substantially axial direction to the wound.

In an embodiment, the panel 12 may have an adhesive component coupled to the top side 16. The adhesive component may be coupled to the entire panel, or specific areas such as the protrusions to ensure consistent and secure contact with the skin of the user.

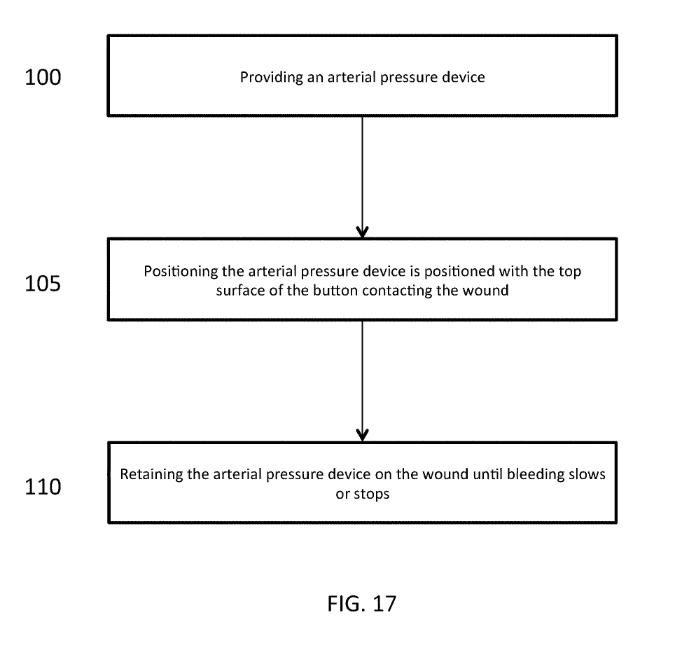

Referring now to FIG. 17, a method of hemostasis in a human comprises the steps of, first in step 100, the user selects a bandage with an arterial pressure device disposed within. The user may optionally place adjuvant materials within the aperture, or select to have multiple apertures on the button of the device. The size, hardness, and resilience of the material may also be selected in combination with the disposed material and number of apertures. In step 105, the user positions a 3-D printed bandage over a wound such that the one or more apertures are positioned directly over the wound. In step 110, the arterial pressure device is held against the wound with an adhesive, bandage, or by mechanical pressure.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A hemostasis device comprising:
a. a panel having a top side and a bottom side, the bottom side having an adhesive layer coupled to the panel;
b. a button integrally molded to the top side of the panel, wherein the button is defined by a 3-dimensional shape;
c. one or more apertures extending through the button and the panel; and
d. a pad, wherein the adhesive layer joins the panel to the pad,
wherein the button is engaged to a wound by a user, and wherein the button is adapted to provide a force vector in a substantially axial direction to the wound.

2. The device of claim 1, wherein at least one of the one or more apertures are configured to receive medical dressing.

3. The device of claim 1, wherein the one or more apertures are configured to receive a hypodermic needle.

4. The device of claim 1, wherein the button has a hemispherical shape.

5. The device of claim 4, wherein the hemispherical button has a flat top.

6. The device of claim 1, wherein the pad further comprises an elevated perimeter, and wherein the elevated perimeter is dimensioned to retain the panel.

7. The device of claim 1, wherein the device is 3-D printed.

8. The device of claim 7, wherein the device has an infill density between 10% and 90%.

9. The device of claim 1, wherein the pad is adapted to bend, wherein bending of the pad increases the force vector in the substantially axial direction to the wound.

10. The device of claim 1, wherein the panel is an ABS plastic of rectangular shape having a surface area greater than a surface area of the button, wherein the panel can flex around the button.

11. A method for imparting hemostasis comprising the steps of:
a. selecting an arterial pressure device;
b. positioning a button having an one or more apertures over a wound; and
c. retaining the arterial pressure device over the wound,
wherein the arterial pressure device imparts a force vector in a substantially axial direction to the wound, wherein the force vector creates a vacuum, and wherein the vacuum draws fluid from a wound through the one or more apertures.

12. The method of claim 11, wherein the arterial pressure device is positioned within a bandage.

13. The method of claim 11, wherein the one or more apertures are configured to receive medical dressing.

14. The device of claim 11, wherein the button has a hemispherical shape.

15. The device of claim 14, wherein the hemispherical button has a flat top.

16. The device of claim 11, wherein the pad further comprises an elevated perimeter, and wherein the elevated perimeter is dimensioned to retain the panel.

17. The device of claim 11, wherein the device is 3-D printed.

18. The device of claim 17, wherein the device has an infill density between 10% and 90%.

19. The device of claim 11, wherein the pad is adapted to bend, wherein bending of the pad increases the force vector in the substantially axial direction to the wound.

20. The device of claim 11, wherein the panel is an ABS plastic of rectangular shape having a surface area greater than a surface area of the button, wherein the panel can flex around the button.
